(19) United States
(12) Patent Application Publication
(10) Pub. No.: US 2014/0227342 A1
(43) Pub. Date: Aug. 14, 2014

(54) ACETYLCHOLINESTERASE INHIBITOR COMPOSITION FOR SEXUAL USE

(71) Applicant: Michael Farber, Pine Brook, NJ (US)

(72) Inventor: Michael Farber, Pine Brook, NJ (US)

(21) Appl. No.: 13/795,758

(22) Filed: Mar. 12, 2013

Related U.S. Application Data
(60) Provisional application No. 61/762,688, filed on Feb. 8, 2013.

(51) Int. Cl. A61K 31/439; A61F 5/41

(52) U.S. Cl. CPC A61K 31/439; A61F 5/41
USPC 424/450; 514/295; 600/39

(57) ABSTRACT

The present document describes a composition containing an effective amount of an acetylcholinesterase inhibitor, a solvent, and a permeation enhancer in a pharmaceutically acceptable carrier, and uses thereof.

### CROSS-REFERENCE TO RELATED APPLICATIONS

This application claims priority under 35 USC §119(e) of U.S. provisional patent application 61/762,688, filed on Feb. 8, 2013 the specification of which is hereby incorporated by reference.

### BACKGROUND

(a) Field

The subject matter disclosed generally relates to compositions and methods for enhancing sexual performance or pleasure by allowing better control of ejaculation and erection, and increasing stimulation of the sexual organs.

(b) Related Prior Art

Human sexual activities refers to the manner in which humans experience and express their sexuality. People engage in a variety of sexual acts from time to time, and for a wide variety of reasons. Sexual activity normally results in sexual arousal and physiological changes in the aroused person that are exemplified by blood flow to the sex organs, therefore optimizing blood flow to these regions should enhance the sexual experience of both parties.

Human sexual activity has sociological, cognitive, emotional, behavioral and biological aspects, including physiological processes such as the reproductive mechanism, the sex drive, sexual intercourse and sexual behavior in all its forms; and personal bonding and shared emotions during sexual activity.

People engage in sexual activity for any of a multitude of possible reasons. Although the primary evolutionary purpose of sexual activity is reproduction, most people engage in sexual activity because of the sexual pleasure they derive from the activity, therefore enhancing blood flow and stimulation to these regions may enhance the sexual pleasure derived from these activities. However, precocious or uncontrolled ejaculation is a highly undesirable effect of being too aroused.

It would be highly desirable to be provided with a means to enhance sexual performance or pleasure, while preventing unwanted ejaculation in male.

Also, it would be highly desirable to be provided with a means to provide better control of ejaculation without diminishing the pleasure derived from intercourse.

Furthermore, it would be highly desirable to be provided with a means to provide better control of ejaculation while maintaining or even enhancing the pleasure derived from intercourse.

### SUMMARY

According to an embodiment, there is provided a composition comprising:
an effective amount of an acetylcholinesterase inhibitor;
a solvent; and
a permeation enhancer;
   in a pharmaceutically acceptable carrier.

The acetylcholinesterase inhibitor may be chosen from delta9-tetrahydrocannabinol (THC), physostigmine, neostigmine, pyridostigmine, ambenonium, demarcarium, rivastigmine, galantamine, caffeine, donepezil, tetrahydroaminoacridine, edrophonium, huperzine A, bis-huperzine A, bis-huperzine B, huperzine A-tacrine, derivatives of huperzine A,

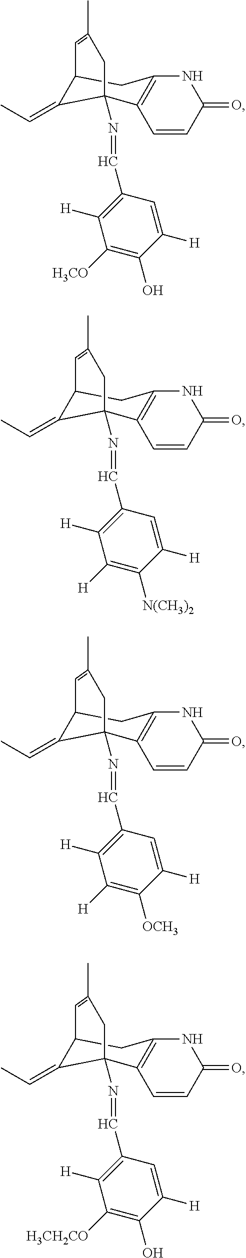

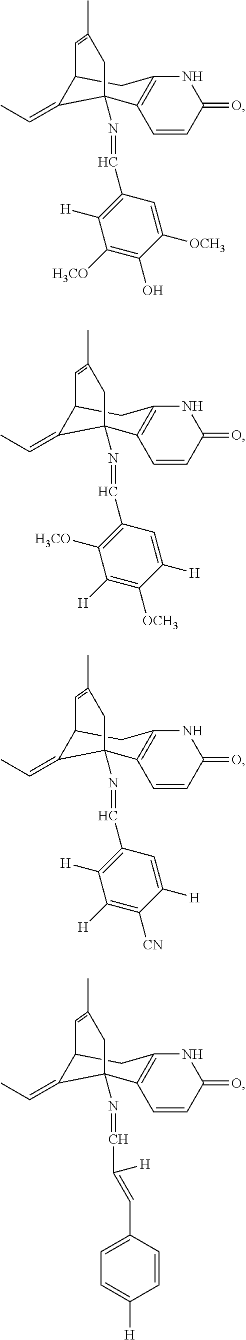 [SMILES: C/C=C1\C2C=C(C)CC1(/N=C/c1cc(OC)c(O)c(OC)c1)c1ccc(=O)[nH]c1C2 ; C/C=C1\C2C=C(C)CC1(/N=C/c1ccc(OC)cc1OC)c1ccc(=O)[nH]c1C2 ; C/C=C1\C2C=C(C)CC1(/N=C/c1ccc(C#N)cc1)c1ccc(=O)[nH]c1C2 ; C/C=C1\C2C=C(C)CC1(N=C/C=C/c1ccccc1)c1ccc(=O)[nH]c1C2]

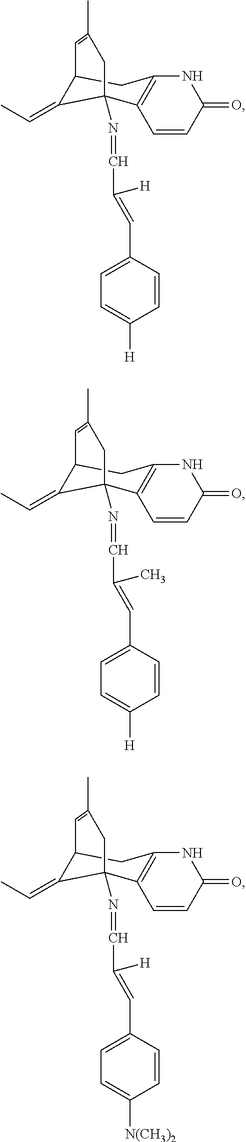 [SMILES: C/C=C1\C2C=C(C)CC1(N=C/C=C/c1ccccc1)c1ccc(=O)[nH]c1C2 ; C/C=C1\C2C=C(C)CC1(N=C/C(C)=C/c1ccccc1)c1ccc(=O)[nH]c1C2 ; C/C=C1\C2C=C(C)CC1(N=C/C=C/c1ccc(N(C)C)cc1)c1ccc(=O)[nH]c1C2]

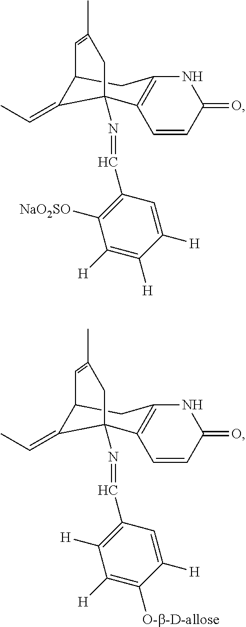

ladostigil, ungeremine, and lactucopicrin.

The acetylcholinesterase inhibitor may be huperzine A.

The acetylcholinesterase inhibitor may be from about 0.001 μg/ml to about 2 μg/ml of the composition.

The cetylcholinesterase inhibitor may be from about 0.1×10⁻⁷% to about 2% (w/w) of the composition.

The acetylcholinesterase inhibitor may be from about 0.1×10⁻⁷% to about 2×10⁻⁴% (w/w) of the composition.

The acetylcholinesterase inhibitor may be formulated as a liposomal formulation.

The composition may be further comprising a cooling agent.

The cooling agent may be chosen from menthol, isopulegole, 3-(1-,2-menthoxy)propane-1,2-diol, p-menthan-3,8-diol, 6-isopropyl-9-methyl-1,4-dioxaspiro-(4,5)-decane-2-methanol, menthyl succinate, a menthyl succinate alkaline earth salt, trimethyl cyclohexanol, N-ethyl-2-isopropyl-5-methylcyclohexane carboxamide, 3-(1-menthoxy)-2-methyl-propan-1,2-diol, mint oil, peppermint oil, wintergreen, menthone, menthone glycerin ketal, menthyl lactate, 1R,2S,5R-2-(5methyl-2(methylethyl)cyclohexyloxy)ethan-1-ol, 1R,2S,5R-3-(5methyl-2(methylethyl)cyclohexyloxy)propan-1-ol, 1R,2S,5R-4-(5-methyl-2(methylethyl)cyclohexyloxy)butanol, spearmint oil or combinations thereof.

The composition may be further comprising a warming agent.

The warming agent may be chosen from vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 12-(1-menthoxy-methyl)-2-(3,4-dihydroxy-phenyl)1,3-dioxolane, 4-(1-menthoxy-methyl)-2-(2-hydroxy-3-methoxy-phenyl)-1,3-dioxolane, 13-(1-menthoxy-methyl)-2-(4-methoxyphenyl)-1,3-dioxolane, 14-(1-menthoxy-methyl)2-(34-methylenedioxy-phenyl)-1,3-dioxolane, hot pepper oil, capsicum oleoresin, ginger olioresin, nonyl acid vanillylamide, or combinations thereof.

The cooling agent may be from about 0.001% to about 10% (w/w) of the composition.

The warming agent may be from about 0.001% to about 10% (w/w) of the composition.

In the composition of the present invention any one of the warming agent or the cooling agent may be from about 0.001% to about 5% (w/w) of the composition.

The permeation enhancer 23-lauryl ether, aprotinin, azone, benzalkonium chloride, cetylpyridinium chloride, cetyltrimethylammonium bromide, cyclodextrin, dextran sulfate, lauric acid, lauric acid/Propylene glycol, lysophosphatidylcholine, menthol, methoxysalicylate, methyloleate, oleic acid, piperine, methyl piperate, a piperine derivative, a methyl piperate derivative, tetrahydropiperine, phosphatidylcholine, polyoxyethylene, polysorbate 80, sodium EDTA, sodium glycocholate, sodium glycodeoxycholate, sodium lauryl sulfate, sodium salicylate, sodium taurocholate, sodium taurodeoxycholate, sodium deoxycholate, a sulfoxide, bile salts, and an alkyl glycoside.

The permeation enhancer may be tetrahydropiperine.

The permeation enhancer may be from about 0.1% to about 0.275% (w/w) of the formulation.

The pharmaceutically acceptable carrier may be a transdermal carrier.

The pharmaceutically acceptable carrier may be one of a lotion, an ointment, a gel, a silicon-based lubricant, a water based lubricant, an oil based lubricant, a hybrid oil-based and silicon-based lubricant.

According to another embodiment, there is provided a method of enhancing sexual performance which comprises contacting a genital tissue of a subject with a composition of the present invention.

According to another embodiment, there may be provided a use of a composition of the present invention for enhancing sexual performance.

According to another embodiment, there is provided a device for stimulating and maintaining a penile erection comprising an elastic sleeve-like member adapted to fit onto a penis, the sleeve-like member having
an interior and an exterior surface; and
a first coating on the interior surface adapted to contact penile tissue and stimulate tumescence, or
a second coating on the exterior coating for stimulating a sexual partner, or combinations thereof;
   wherein the first and second coating comprises a composition of the present invention, in association with a pharmaceutically acceptable carrier.

According to another embodiment, there is provided a method of stimulating and/or maintaining a penile erection comprising:
(a) fitting onto a penis a sleeve-like elastic member of the present invention to stimulate tumescene and produce a penile erection.

The following terms are defined below.

The term “effective amount” is intended to mean the amount of the compound and/or composition that is effective to achieve its intended purpose.

The term “Transdermally absorbed” is intended to mean the delivery of a compound by passage through the skin and into the blood stream.

The term “Transmucosal” is intended to mean the delivery of a compound by passage of the compound through the mucosal tissue and into the blood stream.

The term “Carriers” or “vehicles” are intended to mean carrier materials suitable for compound administration and include any such material known in the art such as, for example, any liquid, lotion, gel, solvent, liquid diluent, solubilizer, or the like, which is non-toxic and which does not interact with any components of the composition in a deleterious manner. Examples include water, glycerol, and propylene glycol.

The term “liposome” is intended to mean an artificially-prepared vesicle composed of a lipid bilayer. The liposome can be used as a vehicle for administration of nutrients and pharmaceutical drugs, and it can be prepared by disrupting biological membranes (such as by sonication). Liposomes are composed of natural phospholipids, and may also contain mixed lipid chains with surfactant properties (e.g., egg phosphatidylethanolamine). The major types of liposomes are the multilamellar vesicle (MLV), the small unilamellar vesicle (SUV), and the large unilamellar vesicle (LUV).

The term “pharmaceutical acceptable carrier” is intended to mean a preservative solution, a saline solution, an isotonic (about 0.9%) saline solution, or about a 5% albumin solution, suspension, sterile water, phosphate buffered saline, and the like. Other buffering agents, dispersing agents, and inert non-toxic substances suitable for delivery to a subject may be included in the compositions of the present invention. The compositions may be solutions, suspensions or any appropriate formulation suitable for administration, and are typically sterile and free of undesirable particulate matter. The compositions may be sterilized by conventional sterilization techniques.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### DETAILED DESCRIPTION

In embodiment there is disclosed a composition comprising:
an acetylcholinesterase inhibitor;
a solvent;
   in a pharmaceutically acceptable carrier.

In another embodiment there is disclosed a method of enhancing sexual performance which comprises contacting a penile tissue of a subject with a composition of the present invention.

In yet another embodiment there is disclosed a device for stimulating and maintaining a penile erection comprising an elastic sleeve-like member adapted to fit onto a penis, the sleeve-like member having
an interior and an exterior surface; and
a first coating on the interior surface adapted to contact penile tissue and stimulate tumescence, or
a second coating on the exterior coating for stimulating a sexual partner, or combinations thereof;
   wherein the first and second coating comprises a composition of the present invention, in association with a pharmaceutically acceptable carrier.

In yet another embodiment there is disclosed a method of stimulating and maintaining a penile erection comprising:
(a) fitting onto a penis a sleeve-like elastic member of the present invention to stimulate tumescene and produce a penile erection.

### Acetylcholinesterase Inhibitors

An acetylcholinesterase inhibitor (often abbreviated AChEI) or anti-cholinesterase is a chemical that inhibits the acetylcholinesterase enzyme from breaking down acetylcholine, thereby increasing both the level and duration of action of the neurotransmitter acetylcholine. Reversible, quasi-irreversible (or pseudo irreversible in some sources) and irreversible inhibitors exist. The acetylcholinesterase inhibitors envisioned to be used in the present invention are the reversible acetylcholinesterase inhibitor.

The transdermal delivery of acetylcholinesterase inhibitor unexpectedly produced highly stimulating effects with respect to sexual arousal, excitement or sensation in a dose dependent manner. The doses required to produce this effect are far below the oral threshold used for supplementation of acetylcholinesterase inhibitor, and the effects are localized to the area of application, such as the genital areas of male and females. However, above a certain concentration threshold, the effects are over stimulating and painful but not skin irritating.

According to an embodiment, the acetylcholinesterase inhibitor may be chosen from delta9-tetrahydrocannabinol (THC), physostigmine, neostigmine, pyridostigmine, ambenonium, demarcarium, rivastigmine, galantamine, caffeine, donepezil, tetrahydroaminoacridine, edrophonium, huperzine A, bis-huperzine A, bis-huperzine B, huperzine A-tacrine, derivatives of huperzine A,

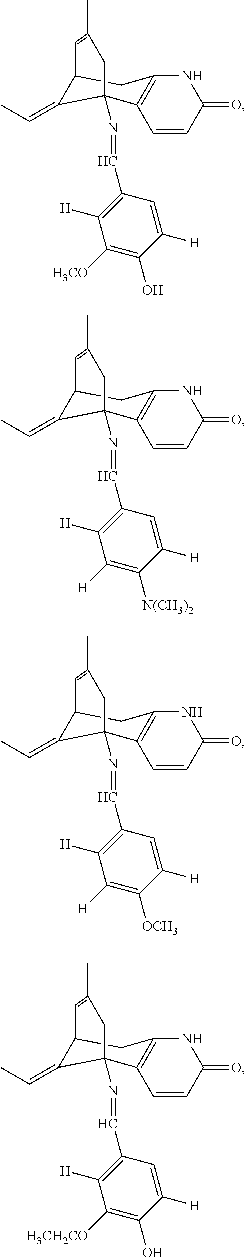

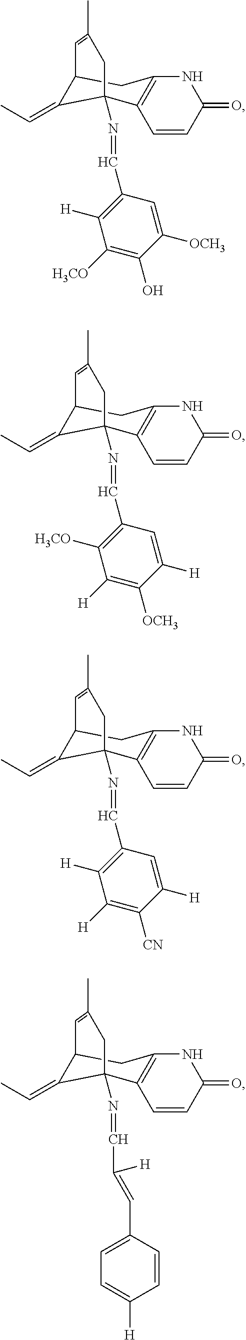 [SMILES: C/C=C1\C2C=C(C)CC1(/N=C/c1cc(OC)c(O)c(OC)c1)c1ccc(=O)[nH]c1C2 ; C/C=C1\C2C=C(C)CC1(/N=C/c1ccc(OC)cc1OC)c1ccc(=O)[nH]c1C2 ; C/C=C1\C2C=C(C)CC1(/N=C/c1ccc(C#N)cc1)c1ccc(=O)[nH]c1C2 ; C/C=C1\C2C=C(C)CC1(N=C/C=C/c1ccccc1)c1ccc(=O)[nH]c1C2]

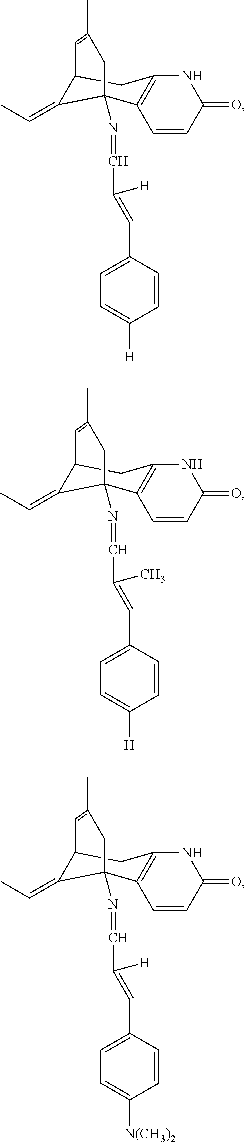 [SMILES: C/C=C1\C2C=C(C)CC1(N=C/C=C/c1ccccc1)c1ccc(=O)[nH]c1C2 ; C/C=C1\C2C=C(C)CC1(N=C/C(C)=C/c1ccccc1)c1ccc(=O)[nH]c1C2 ; C/C=C1\C2C=C(C)CC1(N=C/C=C/c1ccc(N(C)C)cc1)c1ccc(=O)[nH]c1C2]

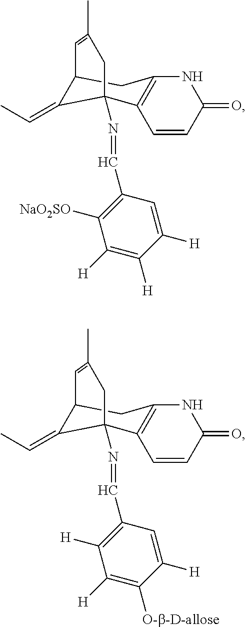

ladostigil, ungeremine, and lactucopicrin. Preferably, the acetylcholinesterase inhibitor is huperzine A.

According to an embodiment, the acetylcholinesterase inhibitor may be used from about 0.001 μg/ml to about 2 μg/ml of the composition. According to another embodiment, the acetylcholinesterase inhibitor may be used from about 0.1×10⁻⁷% to about 2% (w/w) of the composition. According to yet another embodiment, acetylcholinesterase inhibitor may be used from about 0.1×10⁻⁷% to about 2×10⁻⁴% (w/w) of the composition.

According to another embodiment, the acetylcholinesterase inhibitor may be dissolved an equal weight of solvent as the acetylcholinesterase inhibitor. According to another embodiment, the acetylcholinesterase inhibitor may be of synthetic origin or of natural origin, indicating that the compounds are man-made and formed through a chemical process by human agency (synthetic), as opposed to those of natural origin, which may be formed through a chemical process by human agency or obtained from a natural source (e.g. a plant) where the compound(s) may naturally occur.

### Solvents

According to another embodiment, in the composition of the present invention the solvent may be any suitable solvent that is compatible with the skin or mucosal membranes. Non limiting examples include polyethylene glycol (PEG), ethanol, polyhydric alcohols, monohydric alcohols, and combinations thereof. Preferably, the solvent is ethanol.

### Liposome Formulation

According to another embodiment, in the composition of the present invention the acetylcholinesterase inhibitor may be formulated as a liposomal formulation.

According to another embodiment, the formulation of the present invention may comprise a liposome formulation that comprising at least one liposome containing the acetylcholinesterase inhibitor. The liposome formulation may contain more than one type of liposome, each of which may be loaded with one or more different the acetylcholinesterase inhibitor. According to an embodiment, the liposome(s) are loaded with the acetylcholinesterase inhibitor with sufficient amounts to achieve active doses. Without wishing to be bound by theory, the amount of the acetylcholinesterase inhibitor will vary according to the compound selected, and the desired dosage required to obtain the desired biological effect. The person skilled in the art is capable of determining the amount of compound necessary to be loaded into the liposomes in order to achieve the desired amounts in the final dosage forms.

According to an embodiment, unilamellar liposomes may be used to contain water soluble compounds for higher bioavailability and faster absorption.

### Cooling and Warming Agents

According to another embodiment, the composition of the present invention may further comprise a cooling agent, a warming agent, or combinations thereof. The cooling agent may impart sensations of cold, cooling, chilliness and freshness, when applied on skin or mucous membranes. According to another embodiment, the warming agent may impart at least one of the sensations of warmth and warming.

According to an embodiment, the cooling agent may be chosen from menthol, isopulegole, 3-(1-,2-menthoxy)propane-1,2-diol, p-menthan-3,8-diol, 6-isopropyl-9-methyl-1,4-dioxaspiro-(4,5)-decane-2-methanol, menthyl succinate, a menthyl succinate alkaline earth salt, trimethyl cyclohexanol, N-ethyl-2-isopropyl-5-methylcyclohexane carboxamide, 3-(1-menthoxy)-2-methyl-propan-1,2-diol, mint oil, peppermint oil, wintergreen, menthone, menthone glycerin ketal, menthyl lactate, 1R,2S,5R-2-(5methyl-2(methylethyl)cyclohexyloxy)ethan-1-ol, 1R,2S,5R-3-(5methyl-2(methylethyl)cyclohexyloxy)propan-1-ol, 1R,2S,5R-4-(5-methyl-2(methylethyl)cyclohexyloxy)butanol, spearmint oil or combinations thereof.

According to another embodiment, the warming agent may be chosen from vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 12-(1-menthoxy-methyl)-2-(3,4-dihydroxy-phenyl)1,3-dioxolane, 4-(1-menthoxy-methyl)-2-(2-hydroxy-3-methoxy-phenyl)-1,3-dioxolane, 13-(1-menthoxy-methyl)-2-(4-methoxyphenyl)-1,3-dioxolane, 14-(1-menthoxy-methyl)2-(34-methylenedioxy-phenyl)-1,3-dioxolane, hot pepper oil, capsicum oleoresin, ginger olioresin, nonyl acid vanillylamide, or combinations thereof.

According to yet another embodiment, the cooling agent may be from about 0.001% to about 10% (w/w) of the composition. According to yet another embodiment, the warming agent may be from about 0.001% to about 10% (w/w) of the composition. According to yet another embodiment, any one of the warming agent or the cooling agent is from about 0.001% to about 5% (w/w) of the composition.

### Permeation Enhancers

According to another embodiment, the formulation of the present invention may comprise a permeation enhancer. The composition may contain more than one type of permeation enhancer. According to an embodiment, the permeation enhancer(s) may be from about 0.1% to about 0.275% (w/w) of the formulation. Without wishing to be bound by theory, the amount of permeation enhancer will vary according to the permeation enhancer selected, and the desired dosage required to obtain the desired biological beneficial effect. The person skilled in the art is capable of determining the amount of permeation enhancer necessary to achieve the desired amounts in the final composition.

Examples of permeation enhancer include but are not limited to 23-lauryl ether, aprotinin, azone, benzalkonium chloride, cetylpyridinium chloride, cetyltrimethylammonium bromide, cyclodextrin, dextran sulfate, lauric acid, lauric acid/Propylene glycol, lysophosphatidylcholine, menthol, methoxysalicylate, methyloleate, oleic acid, piperine, methyl piperate, a piperine derivative, a methyl piperate derivative, tetrahydropiperine, phosphatidylcholine, polyoxyethylene, polysorbate 80, sodium EDTA, sodium glycocholate, sodium glycodeoxycholate, sodium lauryl sulfate, sodium salicylate, sodium taurocholate, sodium taurodeoxycholate, sodium deoxycholate, a sulfoxide, bile salts, and an alkyl glycoside. The preferred permeation enhancer is tetrahydropiperine (also known as cosmoperine™)

According to another embodiment, the pharmaceutically acceptable carrier is a transdermal carrier. The pharmaceutically acceptable carrier may be one of a lotion, an ointment, a gel, a silicon-based lubricant, a water based lubricant,an oil based lubricant, a hybrid oil-based and silicon-based lubricant.

### Other Ingredients

According to another embodiment, the formulation of the present invention may further comprise additional ingredients known or practiced in the art of sexual stimulation, such as extracts and essences obtained from herbs, plants, leaves, flowers, roots, and other plant parts, fruits, vegetables, and the likes, which may have been prepared by techniques known to the skilled person, such as expression, absorption, maceration, distillation, etc. The essences may also have been artificially created through combination of chemicals and/or oils (e.g. banana, cherry, currant, peach, pineapple, raspberry, strawberry, and vanilla extracts). The other ingredients may also include compounds such as puerarin (from an extract of pueraria lobata, an extract of pueraria phaseoloides, an extract of Radix puerariae, an isolated puerarin compound, or combinations thereof), yohimbine, a yohimbe corynanthe extract, a cnidium monniueri extract, phycopetalum olaccoides extract, eurycoma longefolia extract, turnera diffusa extract, an algae extract, a maca extract (Lepidum meyenii), a nitric oxide donor, a PDE5 inhibitor, a fibroblast growth stimulating compound, a collagen synthesis stimulating compound, or combinations thereof. The composition may comprise the additional ingredient in the quantities required to have a desired effect. For example, the herbal ingredient may be from about 2% (w/v) to about 5% (w/v).

According to another embodiment, there is disclosed a method of enhancing sexual performance which comprises contacting a penile tissue of a subject with a composition of the present invention.

### Device for Penile Stimulation

According to another embodiment, there is disclosed a device for stimulating and maintaining a penile erection comprising an elastic sleeve-like member adapted to fit onto a penis, the sleeve-like member having
an interior and an exterior surface; and
a first coating on the interior surface adapted to contact penile tissue and stimulate tumescence, or
a second coating on the exterior coating for stimulating a sexual partner, or combinations thereof;
   wherein the first and second coating comprises a composition of the present invention in association with a pharmaceutically acceptable carrier.

According to another embodiment, there is disclosed a method of stimulating and maintaining a penile erection comprising:
(a) fitting onto a penis a sleeve-like elastic member of the present invention to stimulate tumescene and produce a penile erection.

The present invention will be more readily understood by referring to the following examples which are given to illustrate the invention rather than to limit its scope.

### EXAMPLE 1

### Penile Stimulating Composition 1

A penile stimulating composition according to the present invention is formulated according to the present recipe:

| Ingredient category | Ingredient name | Amount | |
|---|---|---|---|
| Acetylcholinesterase | Huperzine A | 1 | mg |
| inhibitor | | | |
| Solvent | Ethanol | 1 | ml |
| warming agent | Vanillyl butyl ether | 0.006 | wt/wt |
| Permeation enhancer | Tetrahydropiperine | 0.2% | wt/wt |
| Carrier | Propylene glycol | 500 | ml |
| Carrier | Water | 500 | ml |
| Carrier | glycerol | 200 | ml |

### EXAMPLE 2

### Penile Stimulating Composition 2

A penile stimulating composition according to the present invention is formulated according to the present recipe:

| Ingredient category | Ingredient name | Amount | |
|---|---|---|---|
| Acetylcholinesterase | Huperzine A liposomes* | 1 | mg |
| inhibitor | | | |
| Solvent | Ethanol | 1 | ml |
| warming agent | Vanillyl butyl ether | 0.004% | wt/wt |
| Permeation enhancer | Tetrahydropiperine | 0.175% | wt/wt |
| Carrier | Propylene glycol | 600 | ml |
| Carrier | Water | 600 | ml |
| Carrier | glycerol | 300 | ml |

*The liposome containing composition contains approximately 80% of the Huperzine A of composition 1.

The composition 1, without liposomes affects ejaculation time of male subjects, substantially improving control, and maintaining erection and delaying ejaculation while not preventing stimulation. The composition 2, with liposomes, has been found to have a highly stimulatory effect increasing stimulatory feedbacks during sexual intercourse at a concentration or from approximately 20% less than the composition 1.

### EXAMPLE 3

### Use of Penile Stimulating Compositions for Improving Penile Activity in Healthy Men

18 healthy human men, aged between 26 to 38 years are recruited and assigned randomly into 3 treatment groups: 1) placebo gel (a gel containing all the ingredients of composition #1, but without the acetylcholinesterase inhibitor); 2) composition #1, and 3) composition #2. The subjects were provided with unlabeled tube of the compositions and given instructions to apply the composition to their penis prior to intercourse with their female partner. For each subject, the brachial systolic pressure average versus the diastolic pressure average in mmHg were measure each 30 minutes for: a) 2 hours before use of the compositions or placebo, b) 1 hour during use of the composition or placebo, and c) 1 hour after use of the composition or placebo (and completion of intercourse). All composition penile effects data are provided under testimony of both sexual partners.

TABLE 1
Effect of penile stimulating compositions 1 and 2 in improving penile activity in healthy men

| | | | | Brachial blood pressure | | | Composition penile effect | |
|---|---|---|---|---|---|---|---|---|
| | | | | systolic/diastolic (mm Hg) | | | Penile | |
| Volunteer | Age | Weight | | Before | During | After | sensitivity & | Ejaculation |
| ID | (years) | (Kg) | Composition | Comp. | Comp. | Comp. | rigidity | delay |
| M#1.0 | 32 | 85 | Placebo | 120/71 | 121/72 | 122/70 | = | = |
| M#2.0 | 26 | 75 | Placebo | 119/64 | 120/65 | 120/65 | + | = |
| M#3.0 | 35 | 69 | Placebo | 124/73 | 125/74 | 125/75 | = | + |
| M#4.0 | 37 | 79 | Placebo | 118/71 | 119/70 | 120/72 | = | = |
| M#5.0 | 28 | 65 | Placebo | 116/68 | 117/69 | 118/70 | + | = |
| M#6.0 | 30 | 70 | Placebo | 123/72 | 123/73 | 124/75 | = | = |
| M#1.1 | 27 | 82 | #1 | 123/68 | 123/69 | 124/76 | + | +++ |
| M#2.1 | 29 | 83 | #1 | 118/70 | 120/72 | 123/73 | ++ | +++ |
| M#3.1 | 33 | 77 | #1 | 116/69 | 118/70 | 120/69 | + | +++ |
| M#4.1 | 32 | 67 | #1 | 124/69 | 126/70 | 128/72 | + | +++ |
| M#5.1 | 35 | 85 | #1 | 112/64 | 115/65 | 116/66 | + | +++ |
| M#6.1 | 38 | 69 | #1 | 123/71 | 125/72 | 124/76 | ++ | +++ |
| M#1.2 | 28 | 88 | #2 | 119/68 | 121/69 | 123/71 | ++ | + |
| M#2.2 | 34 | 67 | #2 | 125/76 | 125/72 | 126/73 | ++ | + |
| M#3.2 | 33 | 62 | #2 | 117/65 | 119/67 | 120/68 | + | + |
| M#4.2 | 36 | 75 | #2 | 115/70 | 116/72 | 119/73 | + | ++ |
| M#5.2 | 35 | 72 | #2 | 124/69 | 125/70 | 124/68 | ++ | + |
| M#6.2 | 27 | 76 | #2 | 118/72 | 121/70 | 123/72 | ++ | + |

=: the effect of the composition is equal to that usually observed by the male subject in normal conditions; +: the effect of the composition is slightly higher than that usually observed by the male in normal conditions; ++: the effect of the composition is higher than that usually observed by the male in normal conditions; +++: the effect of the composition is much higher than that usually observed by the male in normal conditions.

The data provided under testimony is generally in line with the expected effects of each composition and placebo. Men who received the placebo composition experienced their normal penile rigidity and delay in ejaculation, although in some cases, the basic gel composition may have enhanced slightly their perception. The composition #1 enhanced penile rigidity and greatly delayed ejaculation above the perceived baseline of the subjects (e.g. for as much as 2 hours). Composition #2 enhanced penile rigidity and ejaculation delay above the perceived baseline of the subjects.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A composition comprising:
an effective amount of an acetylcholinesterase inhibitor;
a solvent; and
a permeation enhancer;
in a pharmaceutically acceptable carrier.

2. The composition of claim 2, wherein said acetylcholinesterase inhibitor is chosen from delta9-tetrahydrocannabinol (THC), physostigmine, neostigmine, pyridostigmine, ambenonium, demarcarium, rivastigmine, galantamine, caffeine, donepezil, tetrahydroaminoacridine, edrophonium, huperzine A, bis-huperzine A, bis-huperzine B, huperzine A-tacrine, a derivative of huperzine A, 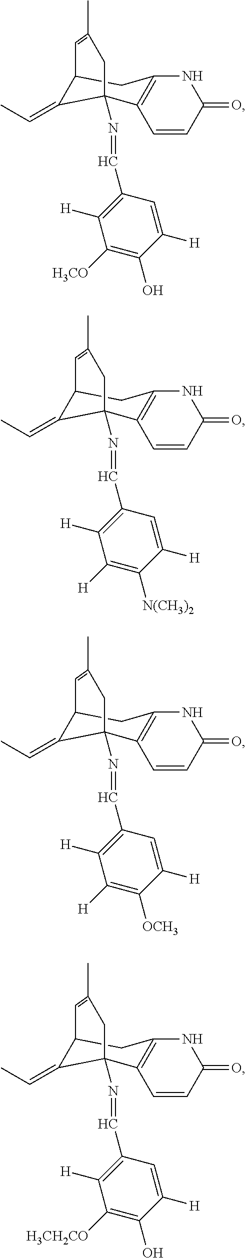 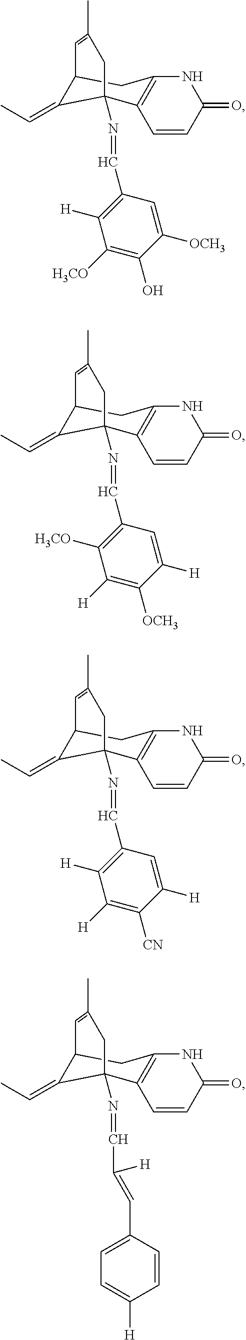 [SMILES: C/C=C1\C2C=C(C)CC1(/N=C/c1cc(OC)c(O)c(OC)c1)c1ccc(=O)[nH]c1C2 ; C/C=C1\C2C=C(C)CC1(/N=C/c1ccc(OC)cc1OC)c1ccc(=O)[nH]c1C2 ; C/C=C1\C2C=C(C)CC1(/N=C/c1ccc(C#N)cc1)c1ccc(=O)[nH]c1C2 ; C/C=C1\C2C=C(C)CC1(N=C/C=C/c1ccccc1)c1ccc(=O)[nH]c1C2] 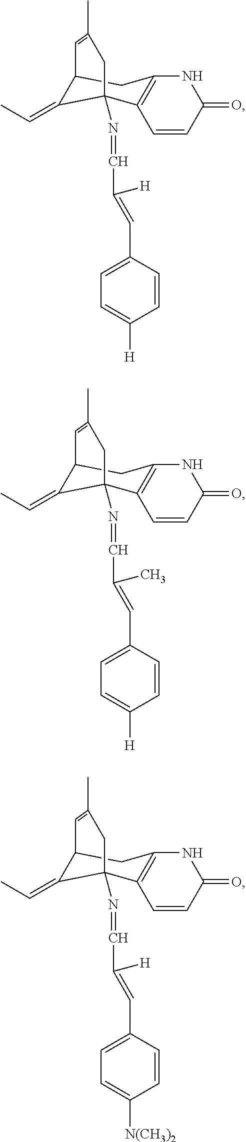 [SMILES: C/C=C1\C2C=C(C)CC1(N=C/C=C/c1ccccc1)c1ccc(=O)[nH]c1C2 ; C/C=C1\C2C=C(C)CC1(N=C/C(C)=C/c1ccccc1)c1ccc(=O)[nH]c1C2 ; C/C=C1\C2C=C(C)CC1(N=C/C=C/c1ccc(N(C)C)cc1)c1ccc(=O)[nH]c1C2] 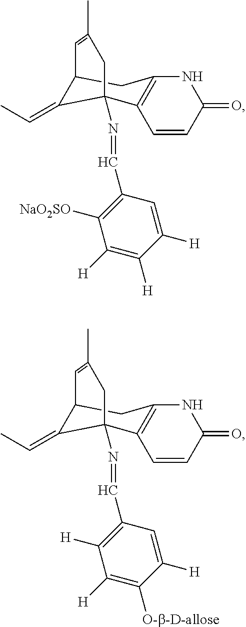
ladostigil, ungeremine, and lactucopicrin.

3. The composition of claim 2, wherein said acetylcholinesterase inhibitor is huperzine A.

4. The composition of claim 1, wherein said acetylcholinesterase inhibitor is from about 0.001 μg/ml to about 2 μg/ml of said composition.

5. The composition of claim 1, wherein said acetylcholinesterase inhibitor is from about 0.1×10⁻⁷% to about 2% of said composition.

6. The composition of claim 5, wherein said acetylcholinesterase inhibitor is from about 0.1×10⁻⁷% to about 2×10⁻⁴% (w/w) of said composition.

7. The composition of claim 1, wherein said acetylcholinesterase inhibitor is formulated as a liposomal formulation.

8. The composition of claim 1, further comprising one of a cooling agent, or a warming agent.

9. The composition of claim 8, wherein said cooling agent is chosen from menthol, isopulegole, 3-(1-,2-menthoxy)propane-1,2-diol, p-menthan-3,8-diol, 6-isopropyl-9-methyl-1,4-dioxaspiro-(4,5)-decane-2-methanol, menthyl succinate, a menthyl succinate alkaline earth salt, trimethyl cyclohexanol, N-ethyl-2-isopropyl-5-methylcyclohexane carboxamide, 3-(1-menthoxy)-2-methyl-propan-1,2-diol, mint oil, peppermint oil, wintergreen, menthone, menthone glycerin ketal, menthyl lactate, 1R,2S,5R-2-(5methyl-2(methylethyl)cyclohexyloxy)ethan-1-ol, 1R,2S,5R-3-(5methyl-2(methylethyl)cyclohexyloxy)propan-1-ol, 1R,2S,5R-4-(5-methyl-2(methylethyl)cyclohexyloxy)butanol, spearmint oil or combinations thereof.

10. The composition of claim 8, wherein said warming agent is chosen from vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 12-(1-menthoxy-methyl)-2-(3,4-dihydroxy-phenyl)1,3-dioxolane, 4-(1-menthoxy-methyl)-2-(2-hydroxy-3-methoxy-phenyl)-1,3-dioxolane, 13-(1-menthoxy-methyl)-2-(4-methoxyphenyl)-1,3-dioxolane, 14-(1-menthoxy-methyl)2-(34-methylenedioxy-phenyl)-1,3-dioxolane, hot pepper oil, capsicum oleoresin, ginger olioresin, nonyl acid vanillylamide, or combinations thereof.

11. The composition of claim 8, wherein any one of said cooling agent or said warming agent is from about 0.001% to about 10% (w/w) of said composition.

12. The composition of claim 11, wherein any one of said warming agent or said cooling agent is from about 0.001% to about 5% (w/w) of said composition.

13. The composition of claim 1, wherein said permeation enhancer 23-lauryl ether, aprotinin, azone, benzalkonium chloride, cetylpyridinium chloride, cetyltrimethylammonium bromide, cyclodextrin, dextran sulfate, lauric acid, lauric acid/Propylene glycol, lysophosphatidylcholine, menthol, methoxysalicylate, methyloleate, oleic acid, piperine, methyl piperate, a piperine derivative, a methyl piperate derivative, tetrahydropiperine, phosphatidylcholine, polyoxyethylene, polysorbate 80, sodium EDTA, sodium glycocholate, sodium glycodeoxycholate, sodium lauryl sulfate, sodium salicylate, sodium taurocholate, sodium taurodeoxycholate, sodium deoxycholate, a sulfoxide, bile salts, and an alkyl glycoside.

14. The composition of claim 13, wherein said permeation enhancer is tetrahydropiperine.

15. The composition of claim 13, wherein said permeation enhancer is from about 0.1% to about 0.275% (w/w) of said formulation.

16. The composition of claim 1, wherein said pharmaceutically acceptable carrier is a transdermal carrier.

17. The composition of claim 1, wherein said pharmaceutically acceptable carrier is one of a lotion, an ointment, a gel, a silicon-based lubricant, a water based lubricant, an oil based lubricant, a hybrid oil-based and silicon-based lubricant.

18. A method of enhancing sexual performance which comprises contacting a genital tissue of a subject with a composition as claimed in claim 1.

19. A device for stimulating and maintaining a penile erection comprising an elastic sleeve-like member adapted to fit onto a penis, said sleeve-like member having
an interior and an exterior surface; and
a first coating on said interior surface adapted to contact penile tissue and stimulate tumescence, or
a second coating on said exterior coating for stimulating a sexual partner, or combinations thereof;
wherein said first and second coating comprises a composition of claim 1, in association with a pharmaceutically acceptable carrier.

20. A method of stimulating and/or maintaining a penile erection comprising:
(a) fitting onto a penis a sleeve-like elastic member of claim 19 to stimulate tumescene and produce a penile erection.
